(19) United States
(12) Patent Application Publication
(10) Pub. No.: US 2011/0213219 A1
(43) Pub. Date: Sep. 1, 2011

(54) Multiple Biomarker Panels to Stratify Disease Severity and Monitor Treatment of Depression

(75) Inventors: John Bilello, Durham, NC (US); Bo Pi, Carlsbad, CA (US)

(73) Assignee: RIDGE DIAGNOSTICS, INC., San Diego, CA (US)

(21) Appl. No.: 13/014,413

(22) Filed: Jan. 26, 2011

Related U.S. Application Data
(60) Provisional application No. 61/298,443, filed on Jan. 26, 2010.

(51) Int. Cl. A61B 5/00

(52) U.S. Cl. 600/301

(57) ABSTRACT

Materials and Methods for stratifying disease severity and for monitoring major depressive disorder are provided.

### CROSS-REFERENCE TO RELATED APPLICATIONS

This application claims benefit of priority from U.S. Provisional Application Ser. No. 61/298,443, filed on Jan. 26, 2010.

### BACKGROUND

1. Technical Field

This document relates to materials and methods for stratifying disease severity and monitoring the effectiveness of treatment in a depressed individual.

2. Background Information

Neuropsychiatric conditions are the world's leader in “years lived with disability” (YLDs), accounting for almost 30% of total YLDs. Unipolar major depressive disorder (MDD) alone accounts for 11% of global YLDs. Several factors, including inaccurate diagnosis, early discontinuation of treatment, and inadequate antidepressant dosing, may contribute to sustained disability and sub-optimal treatment outcomes. For example, nearly one-half of medical outpatients who receive an antidepressant prescription discontinue treatment during the first month. Discontinuation rates within the first three months of treatment can reach 68%, depending on the population studied and the therapeutic agent employed. Efforts to provide optimal treatment to patients with MDD and other neuropsychiatric conditions are often stymied by the traditional reliance upon clinical assessments and the patient's self-reporting of symptoms for diagnosis and for monitoring the efficacy of treatment. Such methods are subjective and often unreliable.

### SUMMARY

Traditional reliance upon clinical assessments and patient interviews for diagnosing depression and establishing a treatment plan can be associated with sub-optimal outcomes for many patients. The ability to determine disease status on an individual basis would permit accurate assessment of a subject's individual disease state. There is a need, however, for reliable methods of diagnosing clinical conditions, and of assessing a subject's disease status or response to treatment. It would be advantageous, therefore, for clinicians and other mental health professionals to use quantitative diagnostic parameters based on physiological measurements to supplement or replace clinical assessments and patient interviews in order to diagnosis depression, accurately stratify disease severity, and monitor the patient's response to treatment. This document is based in part on the identification of such quantitative diagnostic parameters, and on the development of methods for diagnosing depressive disorders, stratifying disease severity, and monitoring a subject's response to treatment.

As described herein, this document provides materials and methods for establishing a baseline diagnosis of depression by developing an algorithm, evaluating multiple parameters, and using the algorithm to determine a set of quantitative diagnostic scores. The approach described herein differs from some of the more traditional approaches to using biomarkers in the construction of an algorithm versus analyzing single markers or groups of markers. As provided herein, algorithms can be used to derive a single value that reflects a particular disease state, prognosis, or response to treatment. Highly multiplexed microarray-based immunological tools can be used to simultaneously measure multiple parameters. In this manner, all results can be derived simultaneously from the same sample and under the same conditions. High-level pattern recognition techniques can be applied using widely available tools such as hierarchical clustering, self-organizing maps, and supervised classification algorithms (e.g., support vector machines, k-nearest neighbors, and neural networks). The latter group of analytical approaches is likely to have substantial value for clinical use of the materials and methods provided herein.

In one aspect, this document features a method for stratifying disease severity in a subject, comprising: (a) providing a numerical value for each of a plurality of analytes relevant to mild, moderate, and severe states of depression, wherein each numerical value corresponds to the level of the analyte in a biological sample from the subject; (b) individually weighting each numerical value in a manner specific to each analyte to obtain a weighted value for each analyte; (c) determining a result value based on an equation that includes each weighted value; (d) comparing the result value to control result values obtained for a normal subject and for subjects having mild, moderate, and severe states of depression, wherein the control result values were determined in a manner comparable to that of the result value; and (e) if the result value is within a predetermined range of control values for no depression, mild depression, moderate depression, or severe depression, classifying the subject having no depression, mild depression, moderate depression, or severe depression, respectively.

The depression can be associated with MDD. An algorithm can be used to calculate a MDD diagnostic score that can be used to support the classification of mild, moderate, and severe states of MDD. The plurality of analytes can include one or more inflammatory biomarkers, one or more neurotrophic biomarkers, one or more metabolic biomarkers, and/or one or more hypothalamic-pituitary-adrenal axis biomarkers. The plurality of analytes can include two or more analytes selected from the group consisting of acylation stimulating protein, adiponectin, adrenocorticotropic hormone, artemin, alpha 1 antitrypsin (A1AT), alpha-2-macroglobin, apolipoprotein C3 (ApoC3), arginine vasopressin, brain-derived neurotrophic factor (BDNF), corticotropin-releasing hormone, C-reactive protein, CD40 ligand, cortisol, epidermal growth factor (EGF), granulocyte colony-stimulating factor, interleukin-1, interleukin-1 receptor agonist, interleukin-6, interleukin-10, interleukin-13, interleukin-18, leptin, macrophage inflammatory protein 1-alpha, myeloperoxidase (MPO), neurotrophin 3, pancreatic polypeptide, plasminogen activator inhibitor-1, prolactin, RANTES, resistin, reelin, S100B, soluble tumor necrosis factor alpha, soluble tumor necrosis factor alpha receptor II (sTNFR2), thyroid stimulating hormone, tumor necrosis factor alpha, or a combination thereof. For example, the plurality of analytes can include cortisol, prolactin, EGF, MPO, BDNF, resistin, sTNFR2, ApoC3, and A1AT. The biological sample can be whole blood, serum, plasma, urine, or cerebrospinal fluid. The subject can be a human. In addition, the method can further include obtaining a measured level of one or more of the plurality of analytes for the biological sample, and the result value can be based at least in part on the measured level.

In another aspect, this document features a method for monitoring treatment of a subject diagnosed with a depressive disorder, comprising (a) providing a first numerical value of each of a plurality of analytes relevant to depression, wherein each first numerical value corresponds to the level of the analyte in a first biological sample from the subject; (b) individually weighting each first numerical value in a manner specific to each analyte to obtain a first weighted value for each analyte; (c) determining a first MDD score based on an equation that includes each first weighted value; (d) providing a second numerical value of each of the plurality of analytes, wherein each second numerical value corresponds to the level of the analyte in a second biological sample from the subject, wherein the second biological sample is obtained after treatment for the depressive disorder; (e) individually weighting each second numerical value in a manner specific to each analyte to obtain a second weighted value for each analyte, with the proviso that the weighting is done in a manner comparable to that in step (b); (f) using the equation to determine a second MDD score after treatment of the subject for the depressive disorder; and (g) comparing the first MDD score to the second MDD score and to a control MDD score or range of MDD scores determined from one or more normal subjects, and classifying the treatment as being effective if the second MDD score is closer than the first MDD score to the control MDD score, or classifying the treatment as not being effective if the second MDD score is not closer than the first MDD score to the control MDD score.

The biological sample can be whole blood, serum, plasma, urine, or cerebrospinal fluid. The second MDD score can be determined days, weeks, or months after treatment for depression. The plurality of analytes can be selected from the group consisting of (a) RANTES, PRL, BDNF, S100B, RES, TNFR, A1A, cortisol, and EGF; (b) RANTES, PRL, BDNF, S100B, RES, TNFR, A1A, and EGF; (c) RANTES, PRL, BDNF, S100B, RES, TNFR, and A1A; (d) S100B, PRL, BDNF, RES, TNFR, and A1A; (e) cortisol, PRL, BDNF, RES, TNFR, and A1A; and (f) BDNF, resistin, TNFRII, and A1A. The subject can be a human. In some case, the method can further include obtaining a measured level of one or more of the plurality of analytes for the first or second biological sample, and the corresponding first or second MDD score can be based at least in part on the measured level.

In another aspect, this document features a method for monitoring treatment of a subject diagnosed with a depressive disorder, comprising (a) providing a first numerical value of each of a plurality of analytes relevant to depression, wherein each first numerical value corresponds to the level of the analyte in a first biological sample from the subject; (b) providing a second numerical value of each of the plurality of analytes, wherein each second numerical value corresponds to the level of the analyte in a second biological sample from the subject, wherein the second biological sample is obtained after treatment for the depressive disorder; (c) individually weighting the first and second numerical values in a manner specific to each analyte to obtain a weighted value for each analyte; (d) determining a monitoring score based on an equation that includes the weighted numerical values; and (e) comparing the monitoring score to a control monitoring score, and classifying the treatment as being effective if the monitoring score is greater than or equal to the control monitoring score, or classifying the treatment as not being effective if the monitoring score is less than the control monitoring score.

The biological sample can be whole blood, serum, plasma, urine, or cerebrospinal fluid. The first biological sample can be obtained from the subject before the start of the treatment, and the second biological sample can be obtained from the subject one to 25 days after start of the treatment. The method can further include providing a third numerical value of each of the plurality of analytes, wherein each third numerical value corresponds to the level of the analyte in a third biological sample from the subject; individually weighting the third numerical values in a manner specific to each analyte to obtain a weighted value for each analyte; and determining the monitoring score based on an equation that includes the first, second, and third weighted numerical values for each analyte. The plurality of analytes can be selected from the group consisting of (a) PRL, BDNF, RES, TNFRII, and A1A; and (b) RANTES, PRL, BDNF, S100B, RES, TNFR, A1A, and EGF.

Unless otherwise defined, all technical and scientific terms used herein have the same meaning as commonly understood by one of ordinary skill in the art to which this disclosure pertains. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a flow diagram showing steps that can be taken to develop a disease-specific biomarker panel for assessing the severity of disease or for diagnostic or prognostic purposes.

FIG. 2 is a flow diagram showing steps that can be taken to develop a diagnostic or prognostic algorithm using a disease-specific biomarker panel.

FIG. 3 is a flow diagram showing steps in an exemplary method for determining a basic diagnostic score.

FIG. 4 is a flow diagram showing exemplary steps for using a diagnostic score to diagnose an individual, to select treatment options, and to monitor and optimize treatment.

FIG. 5 is a hypothetical box whisker plot of marker X levels in the blood of patients prior to and following anti-depressive therapy.

FIG. 6 is a graph plotting the correlation between depression diagnostic scores (MDDSCORE™) and Hamilton Depression Rating Scale (HDRS or HAM-D) scores for a group of normal subjects (filled circles) and a group of MDD patients (open circles).

FIG. 7 is a graph plotting patient HAM-D scores at both 2 and 8 weeks after treatment with the antidepressant Lexapro. A decrease in HAM-D score indicates improvement.

FIG. 8 is a graph plotting the change in depression diagnostic score (MDDSCORE™) in a subset of MDD patients at baseline and after 2 weeks of treatment with Lexapro.

FIG. 9 is a graph plotting the potential for the methods disclosed herein to predict efficacy of treatment at 8 weeks by determining the MDDSCORE™ after 2 weeks of treatment.

FIG. 10 is a flow diagram showing exemplary steps for using an algorithm to monitor treatment outcome in MDD patients.

FIG. 11 is a graph plotting the outcome of a treatment prediction prototype in which biomarker measurements obtained during the first two weeks of treatment were used to calculate a monitoring score to predict the outcome after eight weeks of treatment.

### DETAILED DESCRIPTION

This document is based in part on the identification of methods for establishing a diagnosis or prognosis of depression disorder conditions by developing an algorithm, evaluating (e.g., measuring) multiple parameters, and using the algorithm to determine a set of quantitative diagnostic scores. Algorithms for application of multiple biomarkers from biological samples such as serum or plasma can be developed for stratification of disease severity and identification of disease-specific pharmacodynamic markers. In some embodiments, algorithms for application of multiple biomarkers from biological samples such as, for example, cells, serum, or plasma can be developed for patient stratification, identification of pharmacodynamic markers, and monitoring treatment outcome. As used herein, a “biomarker” is a characteristic that can be objectively measured and evaluated as an indicator of a normal biologic or pathogenic process or pharmacological response to a therapeutic intervention. As used herein, an “analyte” is a substance or chemical constituent that can be objectively measured and determined in an analytical procedure such as immunoassay or mass spectrometry.

### Algorithms

This document provides materials and methods for developing algorithms that incorporate multiple measured parameters and that can be used to determine a quantitative diagnostic score. Algorithms for determining an individual's disease status or response to treatment, for example, can be determined for any clinical condition. The algorithms provided herein can be mathematic functions containing multiple parameters that can be quantified using, for example, medical devices, clinical evaluation scores, or biological, chemical, or physical tests of biological samples. Each mathematic function can be a weight-adjusted expression of the levels of parameters determined to be relevant to a selected clinical condition. Univariate and multivariate analyses can be performed on data collected for each marker using conventional statistical tools (e.g., not limited to: T-tests, PCA, LDA, or binary logistic regression). An algorithm can be applied to generate a set of diagnostic scores. The algorithms generally can be expressed in the format of Formula 1:

Diagnostic score=f(x1,x2,x3,x4,x5 . . . xn) (1)

The diagnostic score is a value that is the diagnostic or prognostic result, “f” is any mathematical function, “n: is any integer (e.g., an integer from 1 to 10,000), and x1, x2, x3, x4, x5 . . . xn are the “n” parameters that are, for example, measurements determined by medical devices, clinical evaluation scores, and/or test results for biological samples (e.g., human biological samples such as blood, serum, plasma, urine, or cerebrospinal fluid).

The parameters of an algorithm can be individually weighted. An example of such an algorithm is expressed in Formula 2:

Diagnostic score=a1*x1+a2*x2−a3*x3+a4*x4−a5*x5 (2)

Here, x1, x2, x3, x4, and x5 can be measurements determined by medical devices, clinical evaluation scores, and/or test results for biological samples (e.g., human biological samples), and a1, a2, a3, a4, and a5 are weight-adjusted factors for x1, x2, x3, x4, and x5, respectively.

A diagnostic score can be used to quantitatively define a medical condition or disease, or the effect of a medical treatment. For example, an algorithm can be used to determine a diagnostic score for a disorder such as depression. In such an embodiment, the degree of depression can be defined based on Formula 1, with the following general formula:

Depression diagnosis score=f(x1,x2,x3,x4,x5 . . . xn)

The depression diagnosis score is a quantitative number that can be used to measure the status or severity of depression in an individual, “f” is any mathematical function, “n” can be any integer (e.g., an integer from 1 to 10,000), and x1, x2, x3, x4, x5 . . . xn are, for example, the “n” parameters that are measurements determined using medical devices, clinical evaluation scores, and/or test results for biological samples (e.g., human biological samples).

In a more general form, multiple diagnostic scores Sm can be generated by applying multiple formulas to specific groupings of biomarker measurements, as illustrated in Formula 3:

Diagnostic scores Sm=Fm(x1, . . . xn) (3)

Multiple scores can be useful, for example, in the identification of specific types and subtypes of depressive disorders and/or associated disorders. In some cases, the depressive disorder is MDD. Multiple scores can also be parameters indicating patient treatment progress or the efficacy of the treatment selected. Diagnostic scores for subtypes of depressive disorders may help aid in the selection or optimization of antidepressants and other pharmaceuticals.

### Building Biomarker Libraries

To determine what parameters are useful for inclusion in a diagnostic algorithm, a biomarker library of analytes can be developed, and individual analytes from the library can be evaluated for inclusion in an algorithm for a particular clinical condition. As a starting point, the library can include analytes generally indicative of inflammation, cellular adhesion, immune responses, or tissue remodeling. In some embodiments (e.g., during initial library development), a library can include a dozen or more markers, a hundred markers, or several hundred markers. For example, a biomarker library can include a few hundred protein analytes. As a biomarker library is built, new markers can be added (e.g., markers specific to individual disease states, and/or markers that are more generalized, such as growth factors). In some cases, a biomarker library can be refined by addition of disease related proteins obtained from discovery research (e.g., using differential display techniques, such as isotope coded affinity tags (ICAT) or mass spectroscopy). In this manner, a library can become increasingly specific to a particular disease state.

The addition of a new protein analyte to a biomarker library can require a purified or recombinant molecule, as well as an appropriate antibody (or antibodies) to capture and detect the new analyte. Addition of a new nucleic acid-based analyte to a biomarker library can require identification of a specific mRNA, as well as probes and detection systems to quantify the expression of that specific RNA. Although discovery of individual “new or novel” biomarkers is not necessary for developing useful algorithms, such markers can be included. Platform technologies that are suitable for multiple analyte detection methods as described herein typically are flexible and open to addition of new analytes. For example, the Luminex multiplex assay system (xMAP; luminexcorp.com on the World Wide Web) is flexible and can be readily configured to incorporate new disease-specific analytes.

While this document indicates that multiplexed detection systems can provide robust and reliable measurement of analytes relevant to diagnosing, stratifying, and monitoring clinical conditions, this does not preclude the use of assays capable of measuring the concentration of individual analytes from the panel (e.g., a series of single analyte ELISAs). Biomarker panels can be expanded and transferred to traditional protein arrays, multiplexed bead platforms or label-free arrays, and algorithms can be developed to support clinicians and clinical research.

Custom antibody array(s) can be designed, developed, and analytically validated for about 25-50 antigens. Initially, a panel of about 5 to 10 (e.g., 5, 6, 7, 8, 9, or 10) analytes can be chosen based on their ability to, for example, distinguish affected from unaffected subjects, or to stratify patients from a defined sample set according to disease severity. An enriched database, however, usually one in which more than 10 significant analytes are measured, can increase the sensitivity and specificity of test algorithms. Other panels can be run in addition to the panel reflecting inflammation and immune response to further define the disease state or sub-classify patients. By way of example, data obtained from measurements of neurotrophic factors can discern patients with alterations in neuroplasticity. Similarly, data obtained from measurements of metabolic factors can discern patients with altered or abnormal metabolic function, and data obtained from measurements of factors of the hypothalamic-pituitary-adrenal (HPA or HTPA) axis can discern patients with alterations of the neuroendocrine system. It is noted that such approaches also can include or be applied to other biological molecules including, without limitation, DNA and RNA.

### Selecting Individual Biomarkers

In the construction of libraries or panels, markers and parameters can be selected by any of a variety of methods. The primary consideration for constructing a disease specific library or panel can be knowledge of a parameter's relevance to the disease. Literature searches or experimentation also can be used to identify other parameters/markers for inclusion. Numerous transcription factors, growth factors, hormones, and other biological molecules are associated with neuropsychiatric disorders. The parameters used to choose analytes or define biomarkers for MDD can be selected from, for example, the functional groupings of inflammatory biomarkers, HPA axis factors, metabolic biomarkers, and neurotrophic factors, including neurotrophins, glial cell-line derived neurotrophic factor family ligands (GFLs), and neuropoietic cytokines In some cases, biomarkers for MDD can be a panel of analytes including one or more of acylation stimulating protein (ASP), adiponectin (ACRP30), adrenocorticotropic hormone (ACTH), artemin (ARTN), alpha 1 antitrypsin (A1AT), alpha-2-macroglobin (A2M), apolipoprotein C3 (apoC3), arginine vasopressin (AVP), brain-derived neurotrophic factor (BDNF), corticotropin-releasing hormone (CRH), C-reactive protein (CRP), CD40 ligand, cortisol, epidermal growth factor (EGF), granulocyte colony-stimulating factor (G-CSF), interleukin-1 (IL-1), interleukin-1 Receptor Agonist (IL-1RA), interleukin-6 (IL-6), interleukin-10 (IL-10), interleukin-13 (IL-13), interleukin-18 (IL-18), leptin, macrophage inflammatory protein 1-alpha (MIP-1α), myeloperoxidase, neurotrophin 3 (NT-3), pancreatic polypeptide, plasminogen activator inhibitor-1, prolactin, RANTES, resistin, reelin (RELN), S100B, soluble tumor necrosis factor alpha (TNF-α), thyroid stimulating hormone (TSH), tumor necrosis factor alpha (TNF-α), or any combination thereof.

In some cases, biomarkers can be factors involved in the inflammatory response. A wide variety of proteins are involved in inflammation, and any one of them is open to a genetic mutation that impairs or otherwise disrupts the normal expression and function of that protein. Inflammation also induces high systemic levels of acute-phase proteins. These proteins include C-reactive protein, serum amyloid A, serum amyloid P, vasopressin, and glucocorticoids, which cause a range of systemic effects. Inflammation also involves release of pro-inflammatory cytokines and chemokines Studies have demonstrated that abnormal functioning of the inflammatory response system disrupts feedback regulation of the immune system, thereby contributing to the development of neuropsychiatric and immunologic disorders. In fact, several medical illnesses that are characterized by chronic inflammatory responses (e.g., rheumatoid arthritis) have been reported to be accompanied by depression. Furthermore, recent evidence has linked elevated levels of inflammatory cytokines with both depression and cachexia, and experiments have shown that introducing cytokines induces depression and cachectic symptoms in both humans and rodents, suggesting that there may be a common etiology at the molecular level. For example, administration of pro-inflammatory cytokines (e.g., in cancer or hepatitis C therapies) can induce “sickness behavior” in animals, which is a pattern of behavioral alterations that is very similar to the behavioral symptoms of depression in humans. Therapeutic agents targeting specific cytokine molecules, such as tumor necrosis factor-alpha, are currently being evaluated for their potential to simultaneously treat both depression and cachexia pharmacologically. In sum, the “Inflammatory Response System (IRS) model of depression” (Maes, Adv. Exp. Med. Biol. 461:25-46 (1999)) proposes that pro-inflammatory cytokines, acting as neuromodulators, represent key factors in mediation of the behavioral, neuroendocrine and neurochemical features of depressive disorders.

In some cases, biomarkers can be neurotrophic factors. Neurotrophic factors are a family of proteins that are responsible for the growth and survival of developing neurons and the maintenance of mature neurons. Most neurotrophic factors belong to one of three families: (1) neurotrophins, (2) glial cell-line derived neurotrophic factor family ligands (GFLs), and (3) neuropoietic cytokines. Each family has its own distinct signaling family, yet the cellular responses elicited often overlap. Neurotrophic factors such as brain-derived neurotrophic factor (BDNF) and its receptor, TrkB, are proteins responsible for the growth and survival of developing neurons and for the maintenance of mature neurons. Neurotrophic factors can promote the initial growth and development of neurons in the CNS and PNS, as well as regrowth of damaged neurons in vitro and in vivo. In addition, these factors often are released by a target tissue in order to guide the growth of developing axons. Studies have suggested that deficits in neurotrophic factor synthesis may be responsible for increased apoptosis in the hippocampus and prefrontal cortex that is associated with the cognitive impairment described in depression.

In some cases, biomarkers can be factors of the HPA axis. The HPA axis, also known as the limbic-hypothalamic-pituitary-adrenal axis (LHPA axis), is a complex set of direct influences and feedback interactions among the hypothalamus (a hollow, funnel-shaped part of the brain), the pituitary gland (a pea-shaped structure located below the hypothalamus), and the adrenal (or suprarenal) glands (small, conical organs on top of the kidneys). Interactions among these organs constitute the HPA axis, a major part of the neuroendocrine system that controls the body's stress response and regulates many body processes, including digestion, the immune system, mood and emotions, sexuality, and energy storage and expenditure. Examples of HPA axis biomarkers include ACTH and cortisol. Cortisol inhibits secretion of corticotropin-releasing hormone (CRH), resulting in feedback inhibition of ACTH secretion. This normal feedback loop may break down when humans are exposed to chronic stress, and may be an underlying cause of depression.

In some cases, biomarkers can be metabolic factors. Metabolic biomarkers are a group of biomarkers that provide insight into metabolic processes in wellness and disease states. Human diseases manifest in complex downstream effects, affecting multiple biochemical pathways. For example, depression and other neuropsychiatric disorders often are associated with metabolic disorders such as diabetes. Consequently, various metabolites and the proteins and hormones controlling metabolic processes can be used for diagnosing depressive disorders such as MDD, stratifying disease severity, and monitoring a subject's response to treatment for the depressive disorder.

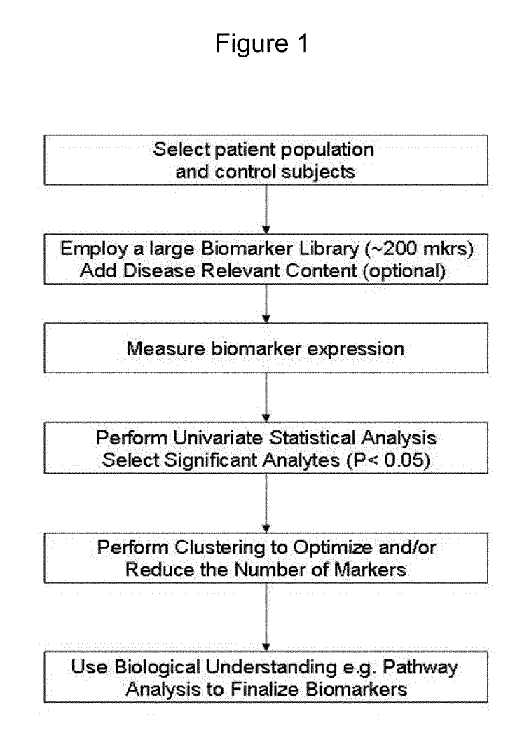

As depicted in the flow diagram of FIG. 1, the process of developing a disease-specific panel of biomarkers can include two statistical approaches: 1) testing the distribution of analytes for association with the disease by univariate analysis; and 2) clustering the analytes into groups using multivariate analysis. In some cases, univariate analysis can be performed to test the distribution of biomarkers for association with MDD, and linear discriminant analysis (LDA) and binary logistic regression can be performed to construct an algorithm to generate a diagnostic score. Univariate analysis explores each variable in a data set separately and identifies the range and central tendency of the values. Multivariate analysis divides the variables into non-overlapping, uni-dimensional clusters. Two or more analytes from each cluster can be selected to design a biomarker or analyte panel for further analyses. The selection typically is based on the statistical strength of the markers and current biological understanding of the disease. For example, analytes chosen according to statistical significance can be subjected to multivariate analysis to identify markers which can distinguish subjects with a clinical condition such as depression from normal populations. Methods for determining statistical significance can be those routinely used in the art including, for example: t-statistics, chi-square statistics, and F-statistics.

In some cases, multivariate analysis can be linear discriminant analysis (LDA), a statistical method used to find the linear combination of features which best separate two or more classes of objects or events. In some cases, multivariate analysis can be principal components analysis (PCA), which is a statistical method that transforms data to a new coordinate system such that the greatest variance by any projection of the data comes to lie on the first coordinate (called the first principal component), the second greatest variance on the second coordinate, and so on. PCA can be used for dimensionality reduction in a data set by retaining those characteristics of the data set that contribute most to its variance, by keeping lower-order principal components and ignoring higher-order ones. Such low-order components often contain the “most important” aspects of the data. In some cases, multivariate analysis can be partial least squares discriminant analysis (PLS-DA), a statistical method used to maximize the separation between groups of variables by rotating PCA components such that a maximum separation among clusters is obtained, and to identify which variables distinguish and separate the clusters.

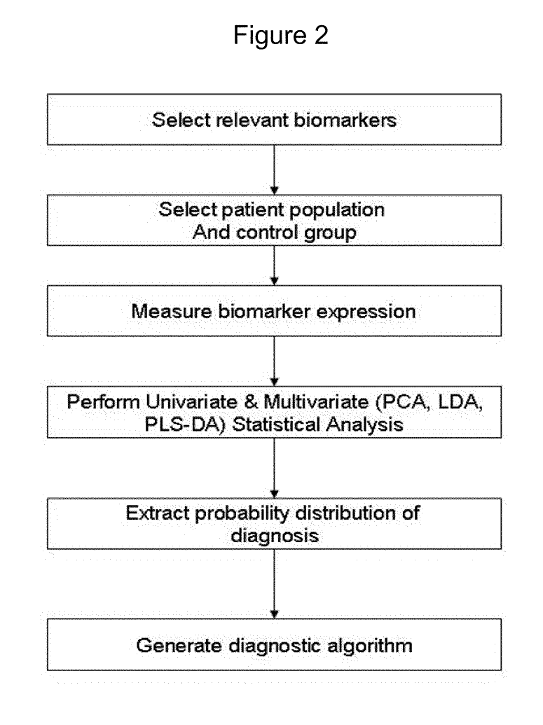

As depicted in the flow diagram of FIG. 2, the selection of relevant biomarkers need not be dependent upon the selection process described in FIG. 1, although the first process is efficient and can provide an experimentally and statistically based selection of markers. The process can be initiated, rather, by a group of biomarkers selected entirely on the basis of hypothesis and currently available data. The selection of a relevant patient population and appropriately matched (e.g., for age, sex, race, and/or BMI) population of normal subjects typically is involved in the process.

### Analyte Measurement

The methods of stratifying disease severity and monitoring a subject's response to treatment for depression provided herein can include determining the levels of a group of biomarkers in a biological sample collected from the subject. An exemplary subject is a human, but subjects can also include animals that are used as models of human disease (e.g., mice, rats, rabbits, dogs, and non-human primates). The group of biomarkers can be specific to a particular disease. For example, a plurality of analytes can form a panel specific to MDD.

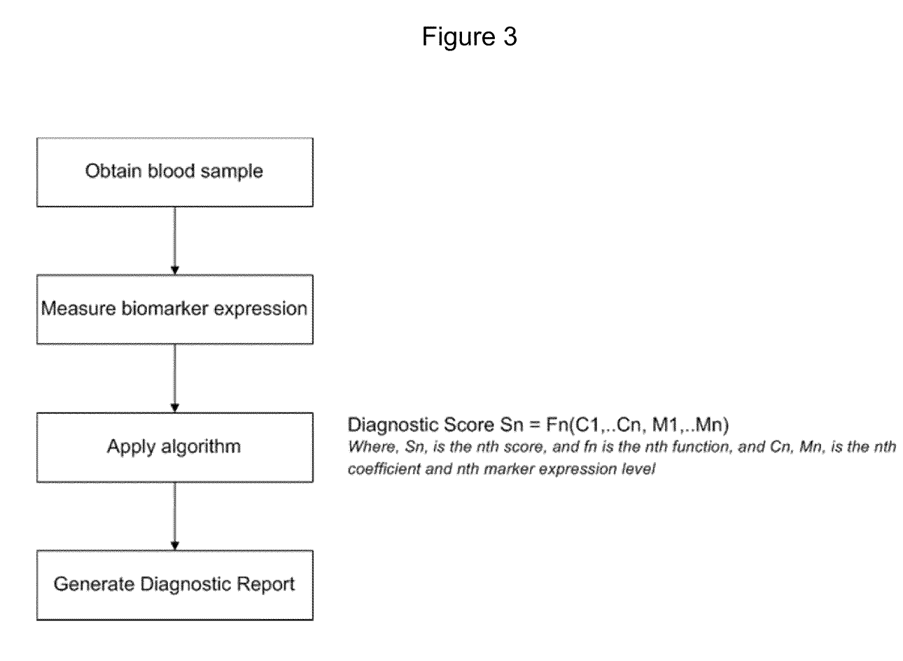

A number of suitable methods can be used to quantify the parameters included in a diagnostic/prognostic algorithm. For example, analyte measurements can be obtained using one or more medical devices or clinical evaluation scores to assess a subject's condition. As depicted in the flow diagram of FIG. 3, the methods provided herein for establishing a diagnostic score can include using tests of biological samples to determine the levels of particular analytes. As used herein, a “biological sample” is a sample that contains cells or cellular material, from which nucleic acids, polypeptides, or other analytes can be obtained. Depending upon the type of analysis being performed, the biological sample can be serum, plasma, or blood cells isolated by standard techniques. Serum and plasma are exemplary biological samples, but other biological samples can be used. For example, specific monoamines can be measured in urine. In addition, depressed patients as a group have been found to excrete greater amounts of catecholamines (CAs) and metabolites in urine than healthy control subjects. Examples of other suitable biological samples include, without limitation, cerebrospinal fluid, pleural fluid, bronchial lavages, sputum, peritoneal fluid, bladder washings, secretions (e.g., breast secretions), oral washings, swabs (e.g., oral swabs), isolated cells, tissue samples, touch preps, and fine-needle aspirates. In some cases, if the biological sample is to be tested immediately, the sample can be maintained at room temperature; otherwise the sample can be refrigerated or frozen (e.g., at −80° C.) prior to assay.

Measurements can be obtained separately for individual parameters, or can be obtained simultaneously for a plurality of parameters. Any suitable platform can be used to obtain parameter measurements. In some cases, biomarker expression levels in a biological sample can be measured using a multi-isotope imaging mass spectrometry (MIMS) instrument or any other suitable technology including, for example, single assays such as ELISA or PCR. Useful platforms for simultaneously quantifying multiple parameters include, for example, those described in U.S. Provisional Application Nos. 60/910,217 and 60/824,471, U.S. Utility application Ser. No. 11/850,550, and PCT Publication No. WO2007/067819, all of which are incorporated herein by reference in their entirety. An example of a useful platform utilizes MIMS label-free assay technology developed by Precision Human Biolaboratories, Inc. (now Ridge Diagnostics, Inc., Research Triangle Park, N.C.). Briefly, local interference at the boundary of a thin film can be the basis for optical detection technologies. For biomolecular interaction analysis, glass chips with an interference layer of SiO₂ can be used as a sensor. Molecules binding at the surface of this layer increase the optical thickness of the interference film, which can be determined as set forth in the applications listed above, for example.

An example of a platform useful for multiplexing is the FDA-approved, flow-based Luminex assay system (xMAP). This multiplex technology uses flow cytometry to detect antibody/peptide/oligonucleotide or receptor tagged and labeled microspheres. In addition, Luminex technology permits multiplexing of up to 100 unique assays within a single sample. Since the system is open in architecture, Luminex can be readily configured to host particular disease panels.

### Methods for Using Diagnostic Scores

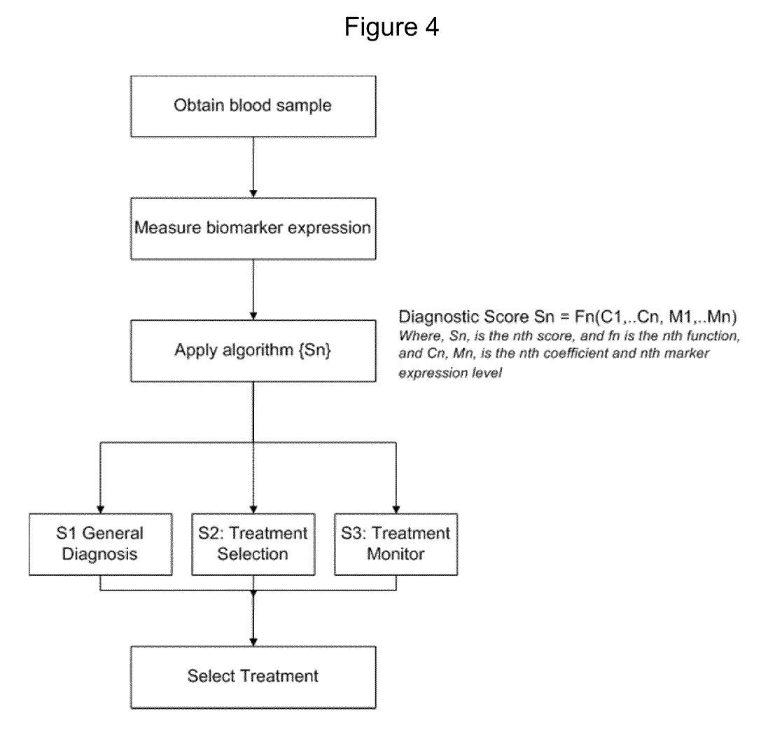

As depicted in the flow diagram of FIG. 4, diagnostic scores can be used to aid in determining diagnosis, stratifying patients, selecting treatments, and monitoring treatment. One or more multiple diagnostic scores can be generated from the expression levels of a set of biomarkers. In this example, multiple biomarkers can be measured from a subject's blood sample, generating three diagnostic scores by the algorithm. In some cases, a single diagnostic score can be sufficient to aid in making a diagnosis and selecting treatment.

Diagnostic scores generated by the methods provided herein can be used to, for example, stratify disease severity. Thus, in some cases, individual analyte levels and/or diagnostic scores determined by the algorithms provided herein can be provided to a clinician for use in diagnosing a subject as having mild, moderate, or severe depression. For example, diagnostic scores generated using the algorithms provided herein can be communicated by research technicians or other professionals who determine the diagnostic scores to clinicians, therapists, or other health-care professionals who will classify a subject as having a particular disease severity based on the particular score, or an increase or decrease in diagnostic score over a period of time. On the basis of these classifications, clinicians, therapists, or other health-care professionals can evaluate and recommend appropriate treatment options, educational programs, and/or other therapies with the goal of optimizing patient care. Diagnoses can be made, for example, using state of the art methodology, or can be made by a single physician or group of physicians with relevant experience with the patient population.

As described herein, a method can include providing a numerical value for each of a plurality of analytes relevant to mild, moderate, and severe states of depression, wherein each numerical value corresponds to the level of the analyte in a biological sample from the subject individually weighting each numerical value in a manner specific to each analyte to obtain a weighted value for each analyte, and determining a result value based on an equation that includes each weighted value. The result value can then be compared to control result values (e.g., values obtained using biological samples from normal subjects and from subjects having mild, moderate, and severe depression), provided that the control result values were determined in a manner comparable to that for the result value. The subject can then be classified as not having depression or as having mild depression, moderate depression, or severe depression, based on where the result value falls as compared to the control values. The method can include using an algorithm to calculate a MDD diagnostic score that can be used to support the classification. The plurality of analytes can include any two or more of those listed in Table 1 herein. In some cases, the plurality can include cortisol, prolactin, EGF, MPO, BDNF, resistin, sTNFR2, ApoC3, and A1AT, for example. In addition, the method can include obtaining a measured level of one or more of the plurality of analytes for the biological sample, wherein the result value is based at least in part on the measured level.

Diagnostic scores also can be used for treatment monitoring. For example, diagnostic scores and/or individual analyte levels can be provided to a clinician for use in establishing or altering a course of treatment for a subject. When a treatment is selected and treatment starts, the subject can be monitored periodically by collecting biological samples at two or more intervals, measuring biomarker levels to generate a diagnostic score corresponding to a given time interval, and comparing diagnostic scores over time. On the basis of these scores and any trends observed with respect to increasing, decreasing, or stabilizing diagnostic scores, a clinician, therapist, or other health-care professional may choose to continue treatment as is, to discontinue treatment, or to adjust the treatment plan with the goal of seeing improvement over time. For example, a decrease in disease severity as determined by a change in diagnostic score can correspond to a patient's positive response to treatment. An increase in disease severity as determined by a change in diagnostic score can indicate failure to respond positively to treatment and/or the need to reevaluate the current treatment plan. A static diagnostic score can correspond to stasis with respect to disease severity. In some cases, movement between disease strata (i.e., mild, moderate, and severe depression) can indicate increasing or decreasing disease severity. In some cases, movement between disease strata can correspond to efficacy of the treatment plan selected for a particular subject or group of subjects.

In some cases, a method can include (a) providing a first numerical value for each of a plurality of analytes relevant to depression, wherein each first numerical value corresponds to the level of the analyte in a first biological sample from the subject, individually weighting each first numerical value in a manner specific to each analyte to obtain a first weighted value for each analyte, and determining a first MDD score based on an equation that includes each first weighted value; and (b) providing a second numerical value of each of the plurality of analytes, wherein each second numerical value corresponds to the level of the analyte in a second biological sample from the subject, wherein the second biological sample is obtained after treatment for the depressive disorder (e.g., treatment for days, weeks, months, or more), individually weighting each second numerical value in a manner specific to each analyte to obtain a second weighted value for each analyte, with the proviso that the weighting is done in a manner comparable to that in step (a), and using the equation to determine a second MDD score after treatment of the subject for the depressive disorder. The first MDD score can be compared to the second MDD score and to a control MDD score or range of MDD scores determined from one or more normal subjects, and the treatment can be classified as effective if the second MDD score is closer than the first MDD score to the control MDD score, or classifying as not effective if the second MDD score is not closer than the first MDD score to the control MDD score.

In some embodiments, an initial blood sample is taken from a subject prior to the start of treatment. The sample optionally can be spun down to separate serum from cells, and stored as PS1 (Patient p draw 1). The subject then can be treated (e.g., with one or more antidepressant drugs) for a length of time, and blood samples can be collected during the course of treatment (e.g., days, weeks, or months after the beginning of treatment). The samples optionally can be spun down, labeled and stored, such that together with the initial sample, there can be multiple samples—PS1, PS2, PS3, etc., depending on the duration of treatment and the frequency of sample collection.

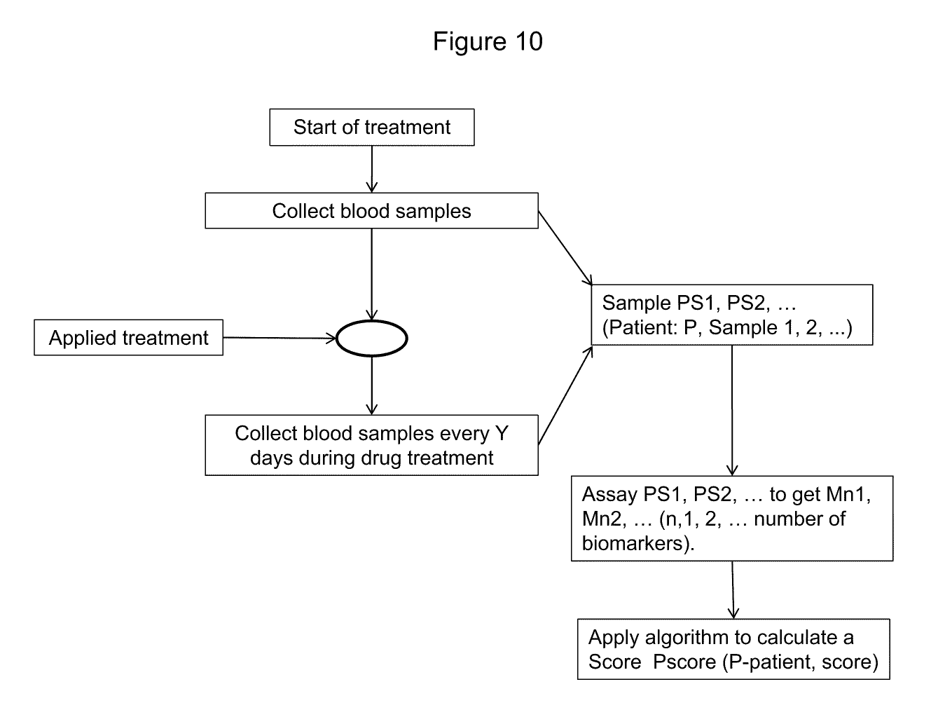

The samples (PS1, PS2, PS3, etc.) for the subject can be assayed to measure the levels of selected biomarkers (Mn1, Mn2, and Mn3=biomarkers n1, n2, and n3; see, e.g., FIG. 10). The marker panel can include biomarkers selected from four major biological systems/pathways (inflammation, HPA axis, metabolic biomarkers, and neurotrophic factors, as described herein). For example, the biomarkers in each pathway include a selection of biomarkers from the list shown in Table 1 (e.g., RANTES, PRL, BDNF, S100B, RES, TNFR, A1A, cortisol, and EGF; RANTES, PRL, BDNF, S100B, RES, TNFR, A1A, and EGF; RANTES, PRL, BDNF, S100B, RES, TNFR, and A1A; S100B, PRL, BDNF, RES, TNFR, and A1A; cortisol, PRL, BDNF, RES, TNFR, and A1A; or BDNF, resistin, TNFRII, and A1A).

A mathematical algorithm can be applied the biomarker measurements to calculate a score that is correlated to the final outcome (e.g., the HAMD score change) at the end of the antidepressant treatment period. The mathematical algorithm can use the specific biomarker changes and the rates of those changes to calculate the score. For example, Mn1, Mn2 (n=1, 2, . . . number of markers) can be used to calculate the score. Alternatively, the changes of (Mn1−Mn2/(Mn1+Mn2) can be used to calculate the score if only two samples are used. In another example, Mn1, Mn2, Mn3 (n=1, 2, 3, . . . number of markers) can be used to calculate the score, and if three samples are collected, the changes of (Mn1−Mn2)/(Mn1+Mn2), (Mn2−Mn3)/(Mn2+Mn3) can be used to calculate the score.

At the end of treatment, the outcome for each patient is known (i.e., whether treatment is successful). This result can be used as an input to optimize the calculation that includes using biomarker measurements (Mn1, Mn2, Mn3, etc.) to predict patient treatment results. Comparing the clinical outcome with the biomarker measurements can optimize generation of a score that maximally correlates to the treatment outcome for a patient treated for depression (e.g., with an antidepressant drug). A control monitoring score can be determined using such a method, and the control score subsequently can be used as a standard to ascertain whether treatment of a subject for depression is effective.

In other embodiments, a method can include at least two (e.g., two, three, four, five, or more than five) numerical values for each of a plurality of analytes relevant to depression, wherein each numerical value corresponds to the level of the analyte in a biological sample from the subject. For example, a first numerical value can be obtained for an analyte in a first biological sample obtained from the subject, a second numerical value can be obtained for the analyte in a second biological sample from the subject, etc. The first biological sample can be obtained before treatment for the depressive disorder, and the second and any subsequent biological samples can be obtained after the onset of treatment (e.g., 12 hours after treatment onset, or one, two, three, four, five, six, seven, 14, 21, or more days after treatment onset). The numerical values can be individually weighted in a manner specific to each analyte, thus giving a weighted value for each analyte, and a “monitoring score” can be determined based on an equation that includes the weighted numerical values. The monitoring score can be compared to a control score, and the success of the treatment can be gauged based on whether the calculated monitoring score is greater than the control score. For example, treatment can be classified as being effective if the monitoring score is greater than or equal to the control monitoring score, or classified as not being effective if the monitoring score is less than the control monitoring score. The plurality of analytes can include any two or more of those listed in Table 1 herein. For example, the plurality of analytes can include PRL, BDNF, RES, TNFRII, and A1A; or RANTES, PRL, BDNF, S100B, RES, TNFR, A1A, and EGF. The control value can be determined from a clinical treatment monitoring study, such that trial data is used to determine a monitoring score that correlates with treatment outcome (e.g., successful treatment). That monitoring score can be established as the control.

After a patient's diagnostic scores are reported, a health-care professional can take one or more actions that can affect patient care. For example, a health-care professional can record a diagnostic or monitoring score in a patient's medical record. In some cases, a health-care professional can record a diagnosis of MDD, or otherwise transform the patient's medical record, to reflect the patient's medical condition. In some cases, a health-care professional can review and evaluate a patient's medical record, and can assess multiple treatment strategies for clinical intervention of a patient's condition.

A health-care professional can initiate or modify treatment for MDD symptoms after receiving information regarding a patient's diagnostic score. In some cases, previous reports of diagnostic scores and/or individual analyte levels can be compared with recently communicated diagnostic scores and/or disease states. On the basis of such comparison, a health-care profession may recommend a change in therapy. In some cases, a health-care professional can enroll a patient in a clinical trial for novel therapeutic intervention of MDD symptoms. In some cases, a health-care professional can elect waiting to begin therapy until the patient's symptoms require clinical intervention.

A health-care professional can communicate diagnostic scores and/or individual analyte levels to a patient or a patient's family. In some cases, a health-care professional can provide a patient and/or a patient's family with information regarding MDD, including treatment options, prognosis, and referrals to specialists, e.g., neurologists and/or counselors. In some cases, a health-care professional can provide a copy of a patient's medical records to communicate diagnostic scores and/or disease states to a specialist.

A research professional can apply information regarding a subject's diagnostic scores and/or disease states to advance MDD research. For example, a researcher can compile data on MDD diagnostic scores with information regarding the efficacy of a drug for treatment of MDD symptoms to identify an effective treatment. In some cases, a research professional can obtain a subject's diagnostic scores and/or individual analyte levels to evaluate a subject's enrollment or continued participation in a research study or clinical trial. A research professional can classify the severity of a subject's condition based on the subject's current or previous diagnostic scores. In some cases, a research professional can communicate a subject's diagnostic scores and/or individual analyte levels to a health-care professional, and/or can refer a subject to a health-care professional for clinical assessment of MDD and treatment of MDD symptoms.

Any appropriate method can be used to communicate information to another person (e.g., a professional), and information can be communicated directly or indirectly. For example, a laboratory technician can input diagnostic scores and/or individual analyte levels into a computer-based record. In some cases, information can be communicated by making a physical alteration to medical or research records. For example, a medical professional can make a permanent notation or flag a medical record for communicating a diagnosis to other health-care professionals reviewing the record. Any type of communication can be used (e.g., mail, e-mail, telephone, and face-to-face interactions). Information also can be communicated to a professional by making that information electronically available to the professional. For example, information can be placed on a computer database such that a health-care professional can access the information. In addition, information can be communicated to a hospital, clinic, or research facility serving as an agent for the professional.

The invention will be further described in the following examples, which do not limit the scope of the invention described in the claims.

### EXAMPLES

### Example 1

### Diagnostic Markers of Depression

Methods as described herein were used to develop an algorithm for determining depression scores that are useful to, for example, diagnose MDD, stratify disease severity, and/or evaluate a patient's response to anti-depressive therapeutics. This systematic, highly parallel, combinatorial approach was proposed to assemble “disease specific signatures” using algorithms as described herein. Two statistical approaches were used for biomarker assessment and algorithm development: (1) univariate analysis of individual analyte levels, and (2) linear discriminant analysis and binary logistic regression for algorithm construction.

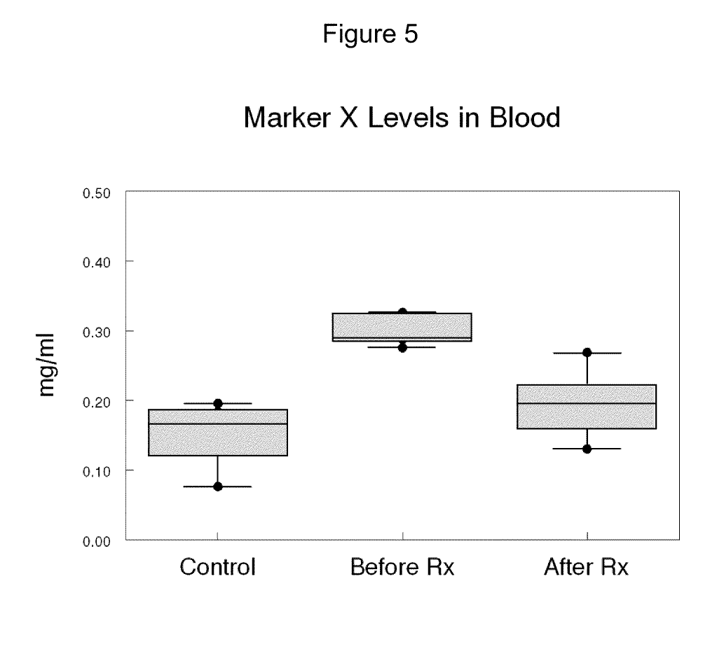

Univariate Analysis of Individual Analyte Levels: Univariate analysis explores each variable in a data set separately. This analysis looks at the range of values, as well as the central tendency of the values, describes the pattern of response to the variable, and describes each variable on its own. By way of example, FIG. 5 shows the distribution of blood levels of marker X in a hypothetical series of six MDD patients before and after treatment. The first point to be made from this graph is that the concentration of marker X was higher in untreated MDD patients as opposed to control subjects. Secondly, after treatment, the levels of marker X in the MDD patients were similar to that of the control. The Student's t-Test was then used to compare two sets of data and to test the hypothesis that a difference in their means was significant. Using the Student's t-Test, serum levels of each of the analytes tested using Luminex multiplex technology (xMAP) were analyzed for comparison of depressed versus normal subjects. Statistical significance was defined as p<0.05. Application of the Student's t-Test to the data, where there were equal numbers of points, showed that the difference in marker X expression between control subjects and patients with MDD was statistically significant (p=0.002), and the difference pre- and post-treatment also was statistically significant (p=0.013). There was no statistically significant difference between the control group and the MDD patients after treatment (p=0.35).

Algorithm Based On Linear Discriminant Analysis: In order to identify the analytes that contribute most to discrimination between classes (e.g., depressed versus normal subjects), a stepwise method of linear discriminant analysis (LDA) from SPSS Statistics v. 11.0 software (SPSS, Inc.) for the Microsoft WINDOWS® operating system was used with the following settings: Wilks' lambda (A) method was used to select analytes that maximize cluster separation and analyte entrance into the model was controlled by its F-value. A large F-value indicates that the level of the particular analyte is different between two groups, and a small F-value (F<1) indicates that there is no difference. In this method, the null hypothesis is rejected for small values of Λ. Thus, the aim was to minimize Λ.

To construct a list of analyte predictors, the F-values for each of the analytes was calculated. Starting with the analyte having the largest F-value (i.e., the analyte that differed most between the two groups), the value of Λ was determined. The analyte with the next largest F-value was then added to the list and Λ was recalculated. If the addition of the second analyte lowered the value of Λ, it was kept in the list of analyte predictors. The process of adding analytes one at a time was repeated until the reduction of Λ no longer occurred.

Cross-validation, a method for testing the robustness of a prediction model, was then carried out. To cross-validate a prediction model, one sample was removed and set aside. The remaining samples were used to build a prediction models based on the pre-selected analyte predictors. A determination was made as to whether the new model was able to predict the one sample not used in building the new model correctly. This process was repeated for all samples, one at a time, to calculate a cumulative cross-validation rate. The final list of analyte predictors was determined by manually entering and removing analytes to maximize the cross-validation rate, using information obtained from the univariate analyses and cross-validations. The final analyte classifier was then defined as the set of analyte predictors that gave the highest cross-validation rate.

Choosing Multiple Biomarkers for Major Depressive Disorder: Serum levels of approximately 100 analytes were tested using Luminex multiplex technology. The Student's t-test was used to compare data for depressed individuals versus normal subjects. Statistical significance was defined as α≦0.05. After the initial study, statistically significant analytes were selected for further study. Table 1 lists the chosen group of biomarkers and indicates how each relates to pathways observed to be altered in subjects with unipolar depression. These analytes were subjected to multivariate analysis (PCA, PLS-DA, and LDA) to identify biomarkers that can be used to distinguish MDD patients from normal populations.

TABLE 1

| Gene Symbol | Gene Name | Pathway |
|---|---|---|
| A1AT | Alpha 1 Antitrypsin | Inflammation |
| A2M | Alpha 2 Macroglobin | Inflammation |
| ApoC3 | Apolipoprotein CIII | Inflammation |
| CD40L | CD40 ligand | Inflammation |
| IL-1 | Interleukin 1 | Inflammation |
| IL-6 | Interleukin 6 | Inflammation |
| IL-13 | Interleukin 13 | Inflammation |
| IL-18 | Interleukin 18 | Inflammation |
| IL-1ra | Interleukin 1 Receptor Antagonist | Inflammation |
| MPO | Myeloperoxidase | Inflammation |
| PAI-1 | Plasminogen activator inhibitor-1 | Inflammation |
| RANTES | RANTES (CCL5) | Inflammation |
| sTNFR2 | Soluble TNF-α Receptor II | Inflammation |
| TNFA | Tumor Necrosis Factor alpha | Inflammation |
| None | Cortisol | HPA axis |
| EGF | Epidermal Growth Factor | HPA axis |
| GCSF | Granulocyte Colony Stimulating | HPA axis |
| | Factor | |
| PPY | Pancreatic Polypeptide | HPA axis |
| ACTH | Adrenocorticotropic hormone | HPA axis |
| AVP | Arginine Vasopressin | HPA axis |
| CRH | corticotropin-releasing hormone | HPA axis |
| ACRP30 | Adiponectin | Metabolic |
| ASP | Acylation Stimulating Protein | Metabolic |
| FABP | Fatty Acid Binding Protein | Metabolic |
| INS | Insulin | Metabolic |
| LEP | Leptin | Metabolic |
| PRL | Prolactin | Metabolic |
| RETN | Resistin | Metabolic |
| None | Testosterone | Metabolic |
| TSH | Thyroid Stimulating Hormone | Metabolic |
| BDNF | Brain-derived neurotrophic factor | \Neurotrophic |
| S100B | S100B | Neurotrophic |
| NTF3 | Neurotrophin 3 | Neurotrophic |
| GDNF | Glial cell line derived neurotrophic | Neurotrophic |
| | factor | |
| ARTN | Artemin | Neurotrophic |

Using nine of the markers listed in Table 1, a diagnostic score was established for each subject based on the following algorithm:

Depression diagnostic score(MDDSCORE™)=f(a1*Cortisol+a2*Prolactin+a3*EGF+a4*MPO+a5*BDNF+a6*Resistin+a7*sTNFR2+a8*ApoC3+a9*A1AT

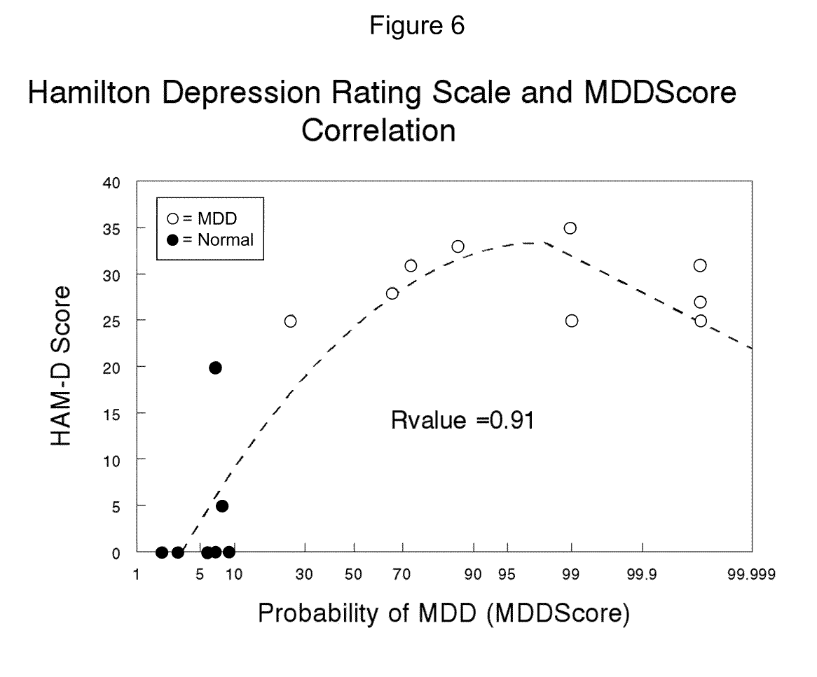

FIG. 6 shows the correlation between the depression diagnostic score and the HAM-D score. The HAM-D is a 21-question multiple choice questionnaire that clinicians can use to rate the severity of a patient's major depression. Support for the view that higher depression rating scale scores do predict a difference in outcome emerged from a review of the U.S. Food and Drug Administration database of 45 clinical trials of antidepressants. This study found that for both the investigational antidepressant [usually a selective serotonin reuptake inhibitor or serotonin-specific reuptake inhibitor (SSRI)] and the active comparator [usually a tricyclic antidepressant (TCA)], the trials with higher mean baseline HAM-D scores were associated with greater reductions in HAM-D scores at the end of a 4- to 8-week trial than trials with a lower mean baseline HAM-D.

### Example 2

### Depression Diagnostic Scores Change Following Drug Therapy

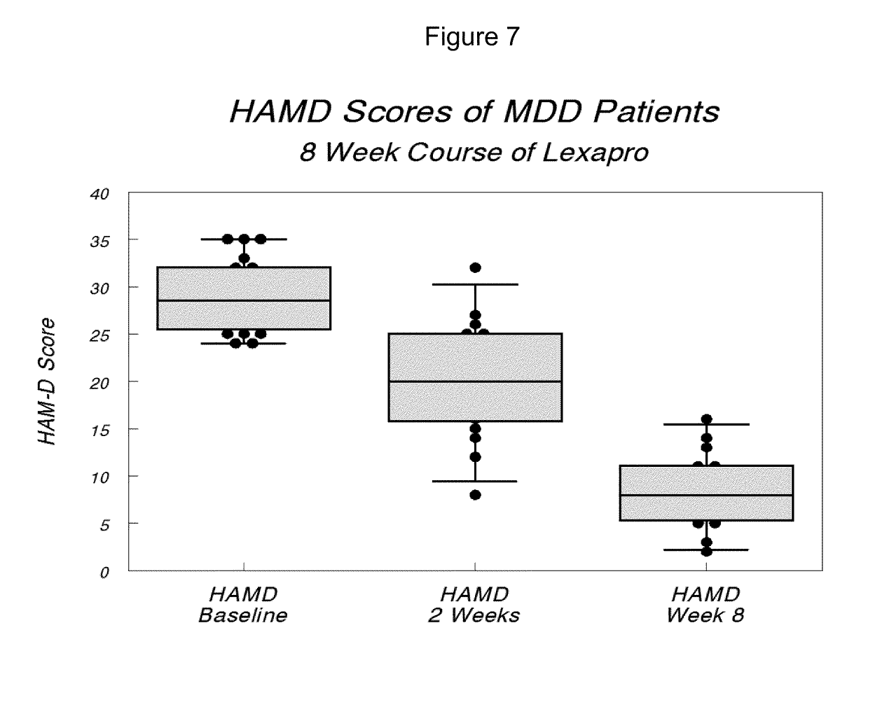

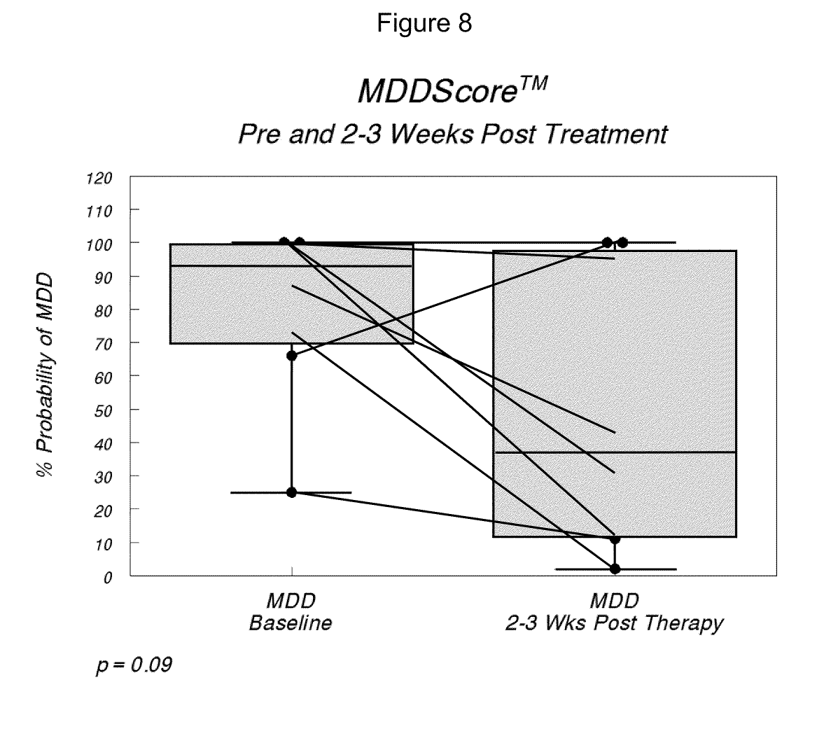

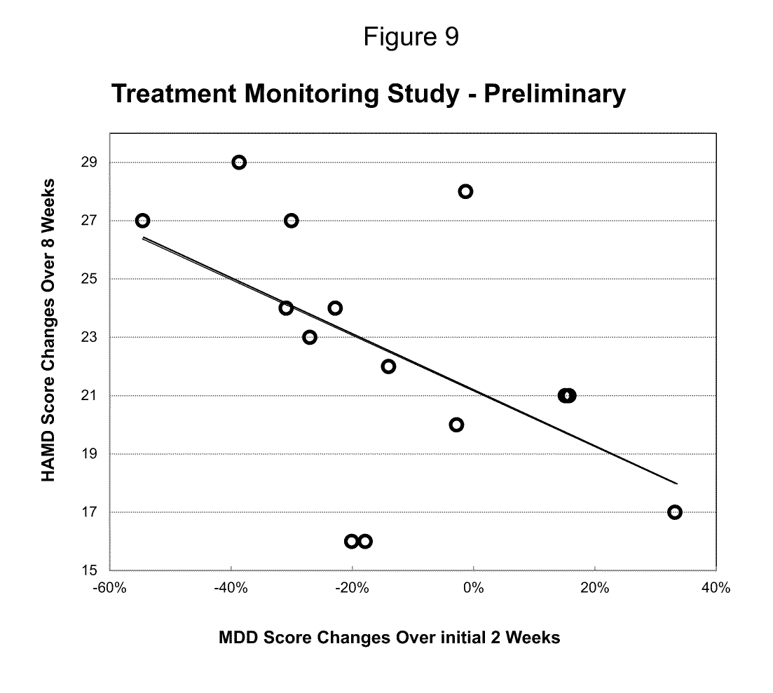

Using the algorithms described herein to establish diagnostic scores, patient populations were stratified according to HAM-D scores above 25. FIG. 7 indicates that patient HAM-D Scores improved (i.e., reduced) at both 2 and 8 weeks after treatment with the antidepressant Lexapro (a SSRI). FIG. 8 shows the change in MDDSCORE™ in a subset of those patients at baseline and after 2 weeks of treatment. FIG. 9 shows the potential for predicting the efficacy of treatment at 8 weeks by determining the MDDSCORE™ after 2 weeks of treatment. These data are indicative of treatment efficacy and demonstrate the utility of MDD diagnostic scores for both patient stratification and treatment monitoring.

### Example 3

### Antidepressant Treatment Monitoring with Multiple Biomarker Measurements

To develop an algorithm for using a panel of biomarker measurements to monitor antidepressant drug treatment, a group of patient candidates was selected for antidepressant drug treatment, and an initial blood sample was taken from each patient. The samples were spun down to separate serum from cells, and stored as PS1 (Patient p draw 1). Each patient was treated with an antidepressant drug (Lexapro®) for eight weeks, and blood samples were collected during the course of treatment. The samples were spun down, labeled and stored.

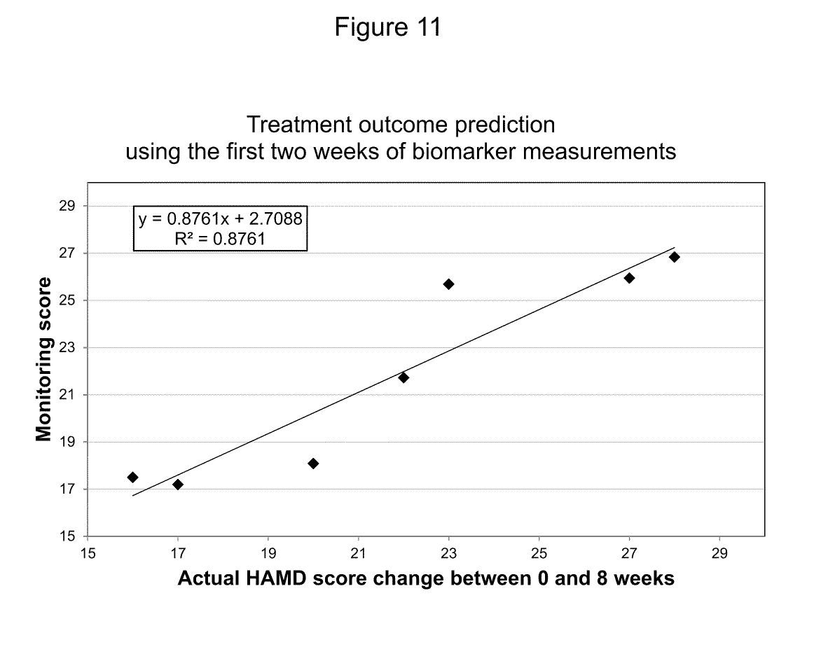

The samples (PS1, PS2, PS3, etc.) for each patient were assayed to measure the levels of five biomarkers—prolactin, BDNF, resistin, TNFRII, and A1A (Mn1, Mn2, and Mn3=biomarkers n1, n2, and n3; FIG. 10). A mathematical algorithm was applied to the biomarker measurements to calculate a monitoring score that was correlated to the final outcome (the HAMD score change) at the end of the antidepressant treatment period. The mathematical algorithm used the specific biomarker changes and the rates of those changes to calculate the score. In particular, the differences in biomarker measurements from two samples (an initial sample and a sample obtained two weeks into treatment) were measured, and a mathematical algorithm was used to calculate a monitoring score to predict the final treatment outcome (indicated by the HAMD score changes between an initial HAMD score and a HAMD score calculated after 8 weeks of treatment). See, FIG. 11.

At end of treatment, the outcome for each patient was known (i.e., whether treatment is successful). This result was used as an input to optimize the calculation that used biomarker measurements (Mn1, Mn2, Mn3, etc.) to predict patient treatment results. Comparing the clinical outcome with the biomarker measurements optimized generation of a monitoring score that maximally correlates to the treatment outcome for a patient treated with an antidepressant drug.

The marker panel included biomarkers selected from four major biological systems/pathways (inflammation, HPA axis, metabolic biomarkers, and neurotrophic factors, as described herein). Other exemplary biomarker panels that are used in the methods described herein include the following:
a biomarker panel including cortisol, PRL, BDNF, RES, TNFR, and A1A; a biomarker panel including S100B, PRL, BDNF, RES, TNFR, and A1A;
a biomarker panel including RANTES, PRL, BDNF, S100B, RES, TNFR, and A1A;
a biomarker panel including RANTES, PRL, BDNF, S100B, RES, TNFR, A1A, and EGF; and
a biomarker panel including RANTES, PRL, BDNF, S100B, RES, TNFR, A1A, cortisol, and EGF.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of stratifying disease severity in a subject, comprising:
(a) providing a numerical value for each of a plurality of analytes relevant to mild, moderate, and severe states of depression, wherein each numerical value corresponds to the level of the analyte in a biological sample from the subject;
(b) individually weighting each numerical value in a manner specific to each analyte to obtain a weighted value for each analyte;
(c) determining a result value based on an equation that includes each weighted value;
(d) comparing the result value to control result values obtained for a normal subject and for subjects having mild, moderate, and severe states of depression, wherein the control result values were determined in a manner comparable to that of the result value; and
(e) if the result value is within a predetermined range of control values for no depression, mild depression, moderate depression, or severe depression, classifying the subject having no depression, mild depression, moderate depression, or severe depression, respectively.

2. The method of claim 1, wherein the depression is associated with major depressive disorder (MDD).

3. The method of claim 2, wherein an algorithm is used to calculate a MDD diagnostic score that can be used to support the classification of mild, moderate, and severe states of MDD.

4. The method of claim 1, wherein the plurality of analytes comprises one or more inflammatory biomarkers.

5. The method of claim 1, wherein the plurality of analytes comprises one or more neurotrophic biomarkers.

6. The method of claim 1, wherein the plurality of analytes comprises one or more metabolic biomarkers.

7. The method of claim 1, wherein the plurality of analytes comprises one or more hypothalamic-pituitary-adrenal axis biomarkers.

8. The method of claim 1, wherein the plurality of analytes comprises two or more analytes selected from the group consisting of acylation stimulating protein, adiponectin, adrenocorticotropic hormone, artemin, alpha 1 antitrypsin (A1AT), alpha-2-macroglobin, apolipoprotein C3 (ApoC3), arginine vasopressin, brain-derived neurotrophic factor (BDNF), corticotropin-releasing hormone, C-reactive protein, CD40 ligand, cortisol, epidermal growth factor (EGF), granulocyte colony-stimulating factor, interleukin-1, interleukin-1 receptor agonist, interleukin-6, interleukin-10, interleukin-13, interleukin-18, leptin, macrophage inflammatory protein 1-alpha, myeloperoxidase (MPO), neurotrophin 3, pancreatic polypeptide, plasminogen activator inhibitor-1, prolactin, RANTES, resistin, reelin, S100B, soluble tumor necrosis factor alpha, soluble tumor necrosis factor alpha receptor II (sTNFR2), thyroid stimulating hormone, tumor necrosis factor alpha, or a combination thereof.

9. The method of claim 1, wherein the plurality of analytes comprises cortisol, prolactin, EGF, MPO, BDNF, resistin, sTNFR2, ApoC3, and A1AT.

10. The method of claim 1, wherein the biological sample is whole blood, serum, plasma, urine, or cerebrospinal fluid.

11. The method of claim 1, wherein the subject is a human.

12. The method of claim 1, further comprising obtaining a measured level of one or more of the plurality of analytes for the biological sample, wherein the result value is based at least in part on the measured level.

13. A method for monitoring treatment of a subject diagnosed with a depressive disorder, comprising:
(a) providing a first numerical value of each of a plurality of analytes relevant to depression, wherein each first numerical value corresponds to the level of the analyte in a first biological sample from the subject;
(b) individually weighting each first numerical value in a manner specific to each analyte to obtain a first weighted value for each analyte;
(c) determining a first MDD score based on an equation that includes each first weighted value;
(d) providing a second numerical value of each of the plurality of analytes, wherein each second numerical value corresponds to the level of the analyte in a second biological sample from the subject, wherein the second biological sample is obtained after treatment for the depressive disorder;
(e) individually weighting each second numerical value in a manner specific to each analyte to obtain a second weighted value for each analyte, with the proviso that the weighting is done in a manner comparable to that in step (b);
(f) using the equation to determine a second MDD score after treatment of the subject for the depressive disorder; and
(g) comparing the first MDD score to the second MDD score and to a control MDD score or range of MDD scores determined from one or more normal subjects, and classifying the treatment as being effective if the second MDD score is closer than the first MDD score to the control MDD score, or classifying the treatment as not being effective if the second MDD score is not closer than the first MDD score to the control MDD score.

14. The method of claim 13, wherein the biological sample is whole blood, serum, plasma, urine, or cerebrospinal fluid.

15. The method of claim 13, wherein the second MDD score is determined days, weeks, or months after treatment for depression.

16. The method of claim 13, wherein the plurality of analytes is selected from the group consisting of:
(a) RANTES, PRL, BDNF, S100B, RES, TNFR, A1A, cortisol, and EGF;
(b) RANTES, PRL, BDNF, S100B, RES, TNFR, A1A, and EGF;
(c) RANTES, PRL, BDNF, S100B, RES, TNFR, and A1A;
(d) S100B, PRL, BDNF, RES, TNFR, and A1A;
(e) cortisol, PRL, BDNF, RES, TNFR, and A1A; and
(f) BDNF, resistin, TNFRII, and A1A.

17. The method of claim 13, wherein the subject is a human.

18. The method of claim 13, further comprising obtaining a measured level of one or more of the plurality of analytes for the first or second biological sample, wherein the corresponding first or second MDD score is based at least in part on the measured level.

19. A method for monitoring treatment of a subject diagnosed with a depressive disorder, comprising:
(a) providing a first numerical value of each of a plurality of analytes relevant to depression, wherein each first numerical value corresponds to the level of the analyte in a first biological sample from the subject;
(b) providing a second numerical value of each of the plurality of analytes, wherein each second numerical value corresponds to the level of the analyte in a second biological sample from the subject, wherein the second biological sample is obtained after treatment for the depressive disorder;
(c) individually weighting the first and second numerical values in a manner specific to each analyte to obtain a weighted value for each analyte;
(d) determining a monitoring score based on an equation that includes the weighted numerical values; and
(e) comparing the monitoring score to a control monitoring score, and classifying the treatment as being effective if the monitoring score is greater than or equal to the control monitoring score, or classifying the treatment as not being effective if the monitoring score is less than the control monitoring score.

20. The method of claim 19, wherein the biological sample is whole blood, serum, plasma, urine, or cerebrospinal fluid.

21. The method of claim 19, wherein the first biological sample is obtained from the subject before the start of the treatment.

22. The method of claim 19, wherein the second biological sample is obtained from the subject one to 25 days after start of the treatment.

23. The method of claim 19, further comprising:
providing a third numerical value of each of the plurality of analytes, wherein each third numerical value corresponds to the level of the analyte in a third biological sample from the subject;
individually weighting the third numerical values in a manner specific to each analyte to obtain a weighted value for each analyte; and
determining the monitoring score based on an equation that includes the first, second, and third weighted numerical values for each analyte.

24. The method of claim 19, wherein the plurality of analytes is selected from the group consisting of:
(a) PRL, BDNF, RES, TNFRII, and A1A; and
(b) RANTES, PRL, BDNF, S100B, RES, TNFR, A1A, and EGF.
